(19) United States
(12) Patent Application Publication
(10) Pub. No.: US 2014/0206939 A1
(43) Pub. Date: Jul. 24, 2014

(54) ENDOSCOPIC AND/OR MEDICAL DEVICE AND COOLING DEVICE FOR AN ENDOSCOPE OR A MEDICAL INSTRUMENT

(71) Applicant: KARL STORZ GMBH & CO. KG, Tuttlingen (DE)

(72) Inventors: Hans-Peter EISELE, Tuttlingen (DE); Peter SCHWARZ, Tuttlingen-Nendingen (DE)

(73) Assignee: KARL STORZ GMBH & CO. KG, Tuttlingen (DE)

(21) Appl. No.: 14/160,891

(22) Filed: Jan. 22, 2014

(30) Foreign Application Priority Data
Jan. 22, 2013 (DE) 10 2013 001 026.8
Jan. 16, 2014 (EP) 14 000 164.5

(51) Int. Cl. A61B 1/12

(52) U.S. Cl. CPC A61B 1/128
USPC 600/156

(57) ABSTRACT

An endoscopic and/or medical device having an endoscope and/or a medical instrument, wherein the endoscope and/or the medical instrument, respectively, has a head piece, wherein in the endoscope and/or in the medical instrument, respectively, at least one heat generating component is arranged, and wherein the head piece has a heat exchange area for discharging the heat generated by the at least one heat generating component to outside the endoscope and/or the medical instrument, respectively. The device has a cooling attachment connectable to the endoscope and/or the medical instrument, respectively, the cooling attachment being configured for absorbing heat from the heat exchange area of the head piece and for dissipating heat via a cooling fluid flowing through the cooling attachment. The invention also relates to a cooling device for an endoscope and/or a medical instrument.

This nonprovisional application claims priority under 35 U.S.C. §119(a) to German Patent Application No. DE 10 2013 001 026.8, which was filed in Germany on Jan. 22, 2013, and which is herein incorporated by reference.

### BACKGROUND OF THE INVENTION

1. Field of the Invention

The invention relates to an endoscopic and/or medical device comprising an endoscope and/or a medical instrument, wherein the endoscope and/or medical instrument, respectively, comprises a head piece, wherein in the endoscope and/or in the medical instrument, respectively, at least one heat generating component is arranged and wherein the head piece has a heat exchange area for discharging the heat generated by the at least one heat generating component to outside the endoscope and/or the medical instrument, respectively. The invention further relates to a cooling device for such an endoscope and/or medical instrument, respectively.

2. Description of the Background Art

Endoscopes for medical or technical applications are distinguished by an elongate shaft configured for being inserted into a hollow space, wherein the shaft contains optical and/or electronic components for capturing an image of a scene in the hollow space and for transmitting the captured image to a display or a viewing device arranged outside the hollow space. In order to sufficiently illuminate the endoscopic scene to be captured, it is known to arrange one or more light sources, for example light emitting diodes (LEDs), close to the distal end of the shaft, i.e. that end of the shaft situated more distant from a user. During operation, however, such a light source generates waste heat which leads to heating of at least the distal end section of the shaft, due to the light source being arranged in the distal end section of the shaft. This heating is undesirable. In particular, heating the surface of the shaft above a temperature of 41° C. may lead to degradation of body tissue contacted by the endoscope in a medical utilization.

Further, an endoscope may exhibit further or other heat sources, such as electronic imaging sensors for capturing an endoscopic image; likewise, during operation of the endoscope waste heat may arise in a light connector and a light guide for transmitting illumination light from the proximal to the distal end section of the endoscope. Medical instruments as well, in particular medical instruments employed in endoscopic surgery, may comprise heat generating components, such as electric or electronic components, motors, or other moving components generating heat due to their power dissipation or by mechanical friction. The heat generating components may be arranged in the distal end section of an endoscope or an endoscopic instrument and may there undesirably cause a temperature rise; on the other hand, the heat generating components may be arranged in other sections of an endoscope or a medical instrument and as well cause an undesirable temperature rise which may, for example, lead to degradation of body tissue or of heat-sensitive components or may heat up a handle, thus impairing the operation of the endoscope or the medical instrument, respectively.

In the publication EP 1 738 679 A2, which corresponds to U.S. Pat. No. 7,914,448, a cooling device has been proposed having a cooling fluid pump, a first heat exchanger for absorbing and discharging thermal energy and a second heat exchanger for discharging the absorbed thermal energy to the atmosphere. The first heat exchanger is located directly near a heat generating electric component, such as an LED, and is arranged in a deflectable part of the endoscope shaft. Instead of providing a second heat exchanger it is also possible to directly discharge the heated cooling fluid. Thereby in particular in medical endoscopes the problem is raised that the passages of the cooling device charged with cooling fluid cannot be completely cleaned or sterilized by autoclaving. Moreover, due to the cooling fluid being routed through the endoscope in principle there is a risk of contaminating the interior of the body of a patient in case of a leakage of the endoscope.

According to EP 2 394 567 A1, which corresponds to US 20110306834, which is incorporated herein by reference, an endoscope is provided that comprises a light source arranged in a distal zone of the shaft, the light source generating waste heat, and a passive cooling which has a heat pipe arranged in the shaft, the heat pipe being thermally coupled to the light source in order to lead away the waste heat in a proximal direction. The at least one heat pipe extends into the head piece of the endoscope in which a heat sink body is arranged which absorbs the waste heat from the heat pipe and discharges the waste heat directly or via a housing of the head piece to the environment.

### SUMMARY OF THE INVENTION

It is therefore an object of the present invention to provide an endoscopic and/or medical device comprising an endoscope and/or a medical instrument as mentioned above as well as a cooling device for such an endoscope and/or medical instrument, wherein more efficient cooling of a heat generating component arranged within the endoscope and/or the medical instrument, respectively, is made possible without necessitating routing cooling fluid supplied from the exterior through channels running through the interior of the endoscope and/or the medical instrument, respectively.

An endoscopic and/or medical device in accordance with an embodiment of the invention can include an endoscope, in particular an endoscope suitable for medical use, and/or a medical instrument, in particular an endoscopic medical instrument; preferably the endoscopic and/or medical device is configured as an endoscopic medical device comprising a medical endoscope or an endoscopic medical instrument. The endoscope and/or the medical instrument, respectively, comprises a head piece having a heat exchange area which, for example, forms part of a surface of a housing of the head piece. In the endoscope and/or in the medical instrument, respectively, at least one heat generating component is arranged, for example one or more LEDs or, for example, an electronic imaging sensor such as a high-resolution CMOS sensor; other optical, mechanical, electric, and electronic components may generate waste heat as well, such as a light connector, a light guide, or a motor or another moving component, generating heat by optic or electric power loss, respectively, or by mechanical friction. The heat exchange area is configured for discharging at least part of the heat generated by the at least one heat generating component to outside the endoscope and/or the medical instrument, respectively.

Within the endoscope and/or the medical instrument, respectively, a heat transmitter may be arranged for transmitting during operation of the endoscope and/or medical instrument, respectively, at least part of the waste heat generated by the at least one heat generating component to the heat exchange area of the head piece. For this purpose the heat transmitter is thermally coupled to the at least one heat generating component, and is thermally coupled to the heat exchange area for transmitting the transmitted heat to the heat exchange area. The heat transmitter may be configured as a wire having a highly heat conductive material or a tube filled with a heat transmitting fluid, in particular as a heat pipe, for example; the heat transmitter may comprise a Peltier element. For transmission of the waste heat generated by the at least one heat generating component, instead of providing a heat transmitter, the at least one heat generating component may be directly thermally coupled to the housing of the head piece or to the heat exchange area.

The endoscopic and/or medical device in accordance with an embodiment of the invention further comprises a cooling attachment which is connectable, in particular detachably connectable, to the endoscope or the medical instrument, respectively, and which is configured for absorbing the heat from the head piece and for discharging heat by means of a cooling fluid flowing through the cooling attachment. For this purpose the cooling attachment may be attachable to the head piece or may be designed for being clipped on the head piece in order to absorb heat from the heat exchange area of the head piece and to discharge the absorbed heat by means of the cooling fluid flowing through the cooling attachment. The cooling fluid is fed to and discharged from the cooling attachment by supply and discharge lines, respectively, for which the cooling attachment may have corresponding connectors.

Due to the cooling attachment being connectable to the head piece for absorbing the waste heat of the at least one heat generating component transmitted to the heat exchange area of the head piece, and due to the cooling attachment being configured for discharging the absorbed heat by means of a cooling fluid flowing through the cooling attachment, on the one hand a particularly efficient cooling is made possible by which an inadmissible temperature rise of the endoscope and/or the medical instrument can be safely avoided and considerable amounts of waste heat can be discharged; therefore active elements such as Peltier elements may also be employed for an especially effective cooling and heat transmission within the endoscope and/or the medical instrument, wherein the waste heat of which elements can be also discharged by the cooling fluid. On the other hand it is not necessitated to this end that the supplied cooling fluid flows through the endoscope and/or medical instrument itself. Therefore, no cleaning of channels in the interior of the endoscope and/or medical instrument, being charged with cooling fluid, is necessitated, and a risk of contamination associated with supplied cooling fluid flowing through the endoscope or medical instrument can be largely avoided.

According to an embodiment of the inventive endoscopic and/or medical device, the device comprises an endoscope and/or medical instrument, respectively, with an elongate shaft at a proximal (i.e. closer to a user) end section of which the head piece having the heat exchange area is arranged. In particular, the shaft is suitable for being inserted into an interior body cavity and may be rigid, semi-flexible or flexible. The at least one heat generating component is arranged in a distal end section of the shaft. A heat transmitter is arranged inside the shaft for transmitting the waste heat generated by the at least one heat generating component during operation of the endoscope and/or medical instrument to the heat exchange area of the head piece. For this purpose the heat transmitter is thermally coupled to the at least one heat generating component and extends into the proximal end section of the shaft, in particular into the head piece, and is thermally coupled to the heat exchange area for transmitting the transmitted heat to the heat exchange area. The heat transmitter may be may be configured as a wire having a highly heat conductive material or a tube filled with a heat transmitting fluid, in particular as a heat pipe; the heat transmitter may comprise a Peltier element. Due to the fact that the head piece featuring the heat exchange area is arranged at a proximal end section of the shaft and that inside the shaft a heat transmitter is arranged for transmitting the waste heat generated by the at least one heat generating component during operation of the endoscope and/or medical instrument to the heat exchange area of the head piece, particularly efficient cooling of a heat generating component arranged within the shaft, in particular in a distal end section of the shaft, is made possible. In this way an inadmissible temperature rise of the shaft, in particular of the distal end section of the shaft of the endoscope and/or the medical instrument can be securely avoided.

The endoscope and/or the medical instrument can be configured to be hermetically sealed and may therefore be designed to be autoclavable. Thus, a particularly safe sterilization is enabled. The endoscope and/or the medical instrument can be designed as a reusable, hermetically sealed endoscope and/or instrument, respectively.

The cooling attachment can be configured as a handle, or forms, together with the head piece, a handle. For this purpose an exterior surface of the cooling attachment may be shaped ergonomically, for example comprising gripping recesses, and may be formed from a material which is optimal for its use as a handle. Further, the cooling attachment may comprise operating elements, for example for controlling a camera arranged in the endoscope or for deflecting an optics or a distal section of the endoscope or the medical instrument. In this way the operation of the endoscope or the medical instrument by a user is facilitated. Due to the heat dissipation being performed by the cooling fluid flowing through the cooling attachment, it is made possible to keep the surface of the cooling attachment on a temperature favorable for use as a handle, independent of a temperature of the heat exchange area, such that the exterior surface of the cooling attachment can be gripped or embraced by a user for handling the endoscope or the medical instrument.

According to an embodiment of the invention the cooling attachment is designed for single use. Therefore costly cleaning and sterilization of the cooling attachment after use in a medical application can be dispensed with, which could be necessitated by through-flow of cooling fluid. In particular it is possible to employ a reusable, preferably a hermetically sealed endoscope or medical instrument with a disposable cooling attachment which at the same time may be configured as a handle. Due to the optical and electronic components and due to the safety requirements valid for a shaft to be inserted into a human body cavity the endoscope or medical instrument contributes the main part of manufacturing cost of the endoscopic and/or medical device, respectively. Thus, an easy and cost-effective use is enabled.

Alternatively, the cooling attachment may be designed for multiple use, wherein for safety reasons a second wall may be provided. The cooling attachment may be configured such that simple cleaning of the channels through which the cooling fluid flows is facilitated. In particular, the endoscope as well as the cooling attachment are designed to be reusable. In this way cost-effective multiple use is made possible. Further, in particular with a reusable cooling attachment, it is preferred for increasing safety that a leakage of the cooling lines running through the cooling attachment is automatically detectable by a sensor system and can be displayed to a user.

The cooling attachment can be configured for being flowed through by a cooling fluid routed in a closed cooling circuit. In such a closed cooling circuit preferably a pump is arranged which conveys the cooling fluid through a first heat exchanger contained in the cooling attachment in order to discharge heat from the cooling attachment. The closed cooling circuit may comprise a second heat exchanger for dissipating the heat discharged by the cooling fluid to the ambient air. Preferably the cooling attachment has connectors for connecting to connection lines by means of which the cooling circuit is closed through the cooling attachment. Length and cross-section of the cooling fluid lines running through the cooling attachment and the conveyor capability of the pump may be adapted to each other such that always a cooling fluid flow sufficient for heat discharge is ensured. Further, a control device may be provided for controlling the pump to generate a cooling fluid flow suitable for discharging the absorbed waste heat; for controlling the cooling fluid flow a temperature sensor may be arranged within the cooling circuit, preferably near the cooling attachment. In this way, in a multiplicity of operating conditions optimal heat dissipation can be ensured.

According to an embodiment of the invention the cooling attachment can be adapted for being flowed through by a cooling fluid under the effect of gravity. The cooling fluid may flow through the cooling attachment in a closed circuit in this case as well, the circulation being driven by convection, wherein the routing of cooling fluid through the first heat exchanger is adapted correspondingly. The length of the lines and the effective cross-section of the flow through the cooling fluid lines running through the cooling attachment may be chosen in an especially preferred manner such that if the cooling fluid is supplied from a sterile water bag under the potential difference which can be realized with customary means in an operating room, sufficient heat discharge can always be ensured; in this case the cooling fluid is not routed in a closed circuit. As the amount of cooling water required for dissipation of the waste heat during a surgical intervention is very small due to the high heat capacity of water, in general the reservoir of a customary sterile water bag of two liters, for example, is sufficient even for long interventions. At the exit of the cooling attachment a flow limiter may be arranged which can be adjusted to the flow rate desired in each case. In this way not only a very simple and fail-safe system is provided, but employing a sterile cooling fluid provides the additional advantage that even in case of a leakage of the cooling attachment or of the supply and/or discharge lines no contamination of the patient can occur.

According to an embodiment of the invention the cooling attachment features a heat exchange area which can be brought at least partially into thermal contact with the heat exchange area of the head piece and which is thermally coupled to a heat exchanger through which the cooling fluid can flow for heat dissipation. In particular the second heat exchange area may be adapted to the first heat exchange area such that by connecting the cooling attachment to the endoscope or the medical instrument both heat exchange areas can be made to directly abut on each other or to be closely adjacent to each other. In the latter case the gap remaining between the heat exchange areas of the head piece and of the cooling attachment may be filled with a thermally conductive material. By the two heat exchange areas being arrangeable to directly abut on or be closely adjacent to each other, in a simple manner an especially intensive heat transfer is made possible.

The heat exchange area of the head piece can be a radially exterior surface of the head piece, with respect to a longitudinal axis of the endoscope or the medical instrument. In this case the cooling attachment may feature a heat exchange area which, in particular, is configured as a corresponding interior surface of a hollow structure of the cooling attachment. The heat exchange area of the head piece can be a lateral area of a cylinder the axis of which is directed parallel to a longitudinal axis of a shaft of the endoscope or the medical instrument, for example, and the heat exchange area of the cooling attachment forms the inner surface of a cylindrical sleeve. The inner radius of the cylindrical sleeve in this case equals to or is slightly larger than the outer radius of the cylindrical area of the head piece configured as the heat exchange area. The heat exchanger of the cooling attachment encompasses the cylindrical heat exchange area of the cooling attachment on its exterior; in turn the heat exchanger may be encompassed by a housing of the cooling attachment configured as a gripping shell of a handle. In this way a simple and stable arrangement is provided permitting efficient heat discharge.

According to an embodiment the heat exchange area of the head piece can form at least a part of a proximal front face of the head piece. Preferably the cooling attachment exhibits a heat exchange area which can be arranged to abut on or to be closely adjacent to the proximal front face of the head piece. In particular the proximal front face of the head piece featuring the heat exchange area of the head piece may be arranged perpendicular to a longitudinal axis of the shaft, and the heat exchange area of the cooling attachment may form a surface which also is arranged perpendicular to the shaft axis and abuts on or is closely adjacent to the front face of the head piece when the cooling attachment is connected to the head piece. In this way in an especially simple manner an effective heat transfer from the head piece to the cooling attachment can be achieved as well.

In a gap remaining between the heat exchange areas of the head piece and the cooling attachment after connecting the cooling attachment to the head piece, a thermally conductive material can be arranged, in particular a permanently elastic thermally conductive material, such as plastic mats filled with thermally conductive particles. The thermally conductive material can be chosen and dimensioned such that in operation of the endoscopic and/or medical device the material is subject to a volume increase due to the transmitted heat and the temperature rise of the heat exchange areas caused in this way; the material may be a phase-change material, for example. In particular the thermally conductive material may be applied to one of the two heat exchange areas, a residual gap remaining between the surfaces of the thermally conductive material and the other of the two heat exchange areas when connecting the cooling attachment to the head piece, the residual gap facilitating connecting the cooling attachment and the head piece to each other. The material can be chosen such and the residual gap is dimensioned such that the material, due to the temperature rise in operation of the device, is subject to such a volume increase that the residual gap is closed. It may also be provided that the volume increase caused by the transmitted heat not only is sufficient to close the residual gap but also to bring about a force closure between the head piece and the cooling attachment via the heat exchange areas and the thermally conductive material arranged in between. In this way a particularly simple and safe operation and at the same time an especially effective heat transfer is facilitated.

According to an embodiment at least one volume variable element which can be filled with the cooling fluid, such as a balloon, is arranged on a surface of the cooling attachment that faces the heat exchange area of the head piece after the cooling attachment and the head piece have been connected to each other. In this case a gap remains between the surface of the cooling attachment and the heat exchange area, the gap being filled by the volume variable element only partially when it is not filled with cooling fluid. Thus there is not impediment in connecting the cooling attachment to the head piece, such as shifting a sleeve of the cooling attachment over a cylindrically shaped heat exchange area of the head piece. In operation of the device the at least one volume variable element is filled with cooling fluid due to the pressure generated by a cooling fluid pump or by gravity, thus being firmly pressed onto the heat exchange area of the head piece. In this case it may be sufficient that cooling fluid flows only through the at least one volume variable element which can be filled with the cooling fluid, without an additional heat exchanger being present in the cooling attachment. The heat transfer from the head piece to the cooling fluid takes place in this case through the wall of the at least one volume variable element, for example through the thin wall of a balloon. Thus, in a simple way an especially effective heat transfer is achievable as well.

In a further embodiment of the invention the cooling attachment is configured such that by connecting the cooling attachment to the head piece a space sealed in a fluid-tight manner can be formed between the heat exchange area of the head piece and a surface of the cooling attachment is formed, wherein cooling fluid can flow through the sealed space. For this purpose a gap may be provided between the heat exchange area of the head piece and the surface of the cooling attachment, the gap being enclosed by one or more seals. In this way a direct and thus particularly efficient heat transfer from the heat exchange area of the head piece to the cooling fluid can be achieved. In this case use of a sterile cooling fluid is especially advantageous in order to avoid a contamination of the patient even in case of a possible leakage of the one or more seals.

In an embodiment, the cooling attachment can be lockably connectable to the head piece. For this purpose, for example, a lock such as a bayonet or a screw joint or a detent connection may be provided. In this way not only secure fixation of the cooling attachment to the head piece is facilitated and thus safe handling of the endoscope or the medical instrument by means of a cooling attachment configured as a handle, for example, but in addition the cooling attachment may be fixable to the head piece being pressed on the head piece. Especially in that case that the heat exchange area is arranged on a proximal front face of the head piece, thus a particularly effective heat transfer is made possible.

According to an embodiment of the invention the head piece and the cooling attachment have cooperating electric plug connector elements which are configured such that by connecting the cooling attachment to the head piece an electric connection between the head piece and the cooling attachment is established. If the connection is formed by a movement in an axial direction of the shaft, for example in that case that the heat exchange area of the head piece forms an axially directed cylinder and the cooling attachment is configured as a sleeve that can be shifted upon the cylinder, the plug connector elements are adapted configured for being stuck together in axial direction. Preferably the cooling attachment has electric connector lines or connectors for an electric connection to a supply apparatus of the endoscopic and/or medical device. Thus, by connecting the cooling attachment to the endoscope or the medical instrument, in the same work step the electric connection for electric supply of the endoscope and/or the medical instrument, respectively, is established, as well as, possibly, an electric connection for transmitting a captured endoscopic image to a display device.

In accordance with an embodiment of the invention a cooling device for a generic endoscopic and/or medical device is designed as a cooling attachment that is connectable to the endoscope and/or medical instrument, respectively, for absorbing heat from the heat exchange area of the head piece and that is configured for dissipating heat by means of a cooling fluid flowing through the cooling attachment. In particular the cooling attachment has the above described features of the cooling attachment. In this way a cooling device is provided that can be handled easily and that permits efficient dissipation of the waste heat generated in an endoscope and/or medical instrument of the kind mentioned, respectively.

Further, the invention relates to a medical instrument in which at least one heat generating component can be arranged and that comprises a head piece having a heat exchange area. Within the medical instrument a heat transmitter may be arranged for transmitting at least part of the waste heat generated by the at least one heat generating component during operation of the medical instrument. The medical instrument is configured to be connected to a cooling attachment as described above for absorbing heat from the head piece and for dissipating heat by means of a cooling fluid flowing through the cooling attachment. A medical instrument according to the invention in particular has the above described features of the medical instrument.

It is to be understood that the above mentioned features as well as the features to be explained below may not only be employed in the combinations mentioned, but also in other combinations or in isolation without leaving the scope of the present invention.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus, are not limitive of the present invention, and wherein:

FIG. 1 is a schematic basic depiction of an endoscopic and/or medical device in accordance with the invention in a longitudinal sectional view;

FIG. 2 is a longitudinal section of a variation of the device in a detailed view;

FIG. 3 is a cross-sectional view through the sleeve of the cooling attachment shown in FIG. 2;

FIG. 4 is a longitudinal sectional view of a head piece and a cooling attachment;

FIG. 5 is a variation of the embodiment of the invention.

### DETAILED DESCRIPTION

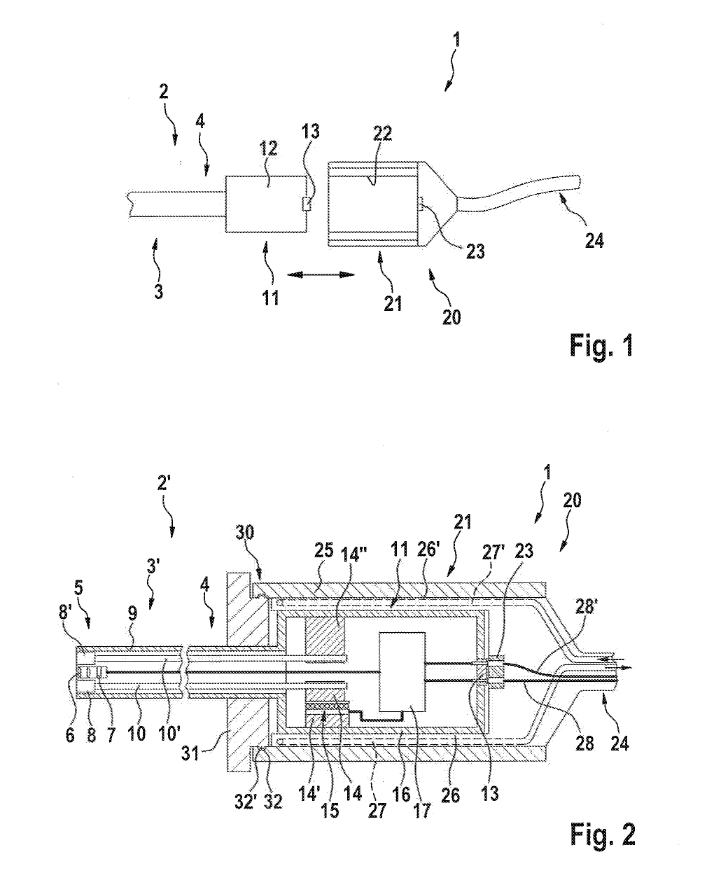

As illustrated schematically in FIG. 1, a medical and/or endoscopic device 1 according to a first embodiment of the invention comprises an endoscope or a medical instrument 2 comprising a distal section 3 shown only symbolically in FIG. 1, and a head piece 11 arranged at its proximal end 4. The head piece 11 is shaped cylindrically with an exterior side 12 forming a cylindrical surface. The device 1 further comprises a cooling attachment 20 designed as a sleeve-like plug and comprising a sleeve 21, the interior side 22 of which cooperates with the exterior side 12 of the head piece 11 for heat exchange. The sleeve 21 can be shifted upon the head piece 11 by an axial movement directed in the direction of the arrow, thus bringing the interior side 22 of the sleeve 21 into contact with the exterior side 12 of the head piece 11 or at least into a situation closely adjacent to the latter. Further, in FIG. 1 the plug connector elements 13, 23 are shown symbolically which cooperate in shifting the sleeve 21 upon the head piece 11 for forming an electric connection. Further, in FIG. 1 a supply line 24 is shown which includes electric lines and lines for the cooling fluid that flows through the cooling attachment 20.

In the sectional view shown in FIG. 2 a first variation of the device shown in FIG. 1 is illustrated. In this variation the device 1 comprises an endoscope 2′ and a cooling attachment 20. The endoscope 2′ has an elongate shaft 3′, wherein at or near the distal end 5 of the shaft 3′ electrical and optical components are arranged, in particular an objective lens 6 for generating an image of an endoscopic scene to be observed on an imaging sensor 7, and two LEDs 8, 8′ for illuminating the endoscopic scene to be observed. In operation the LEDs 8, 8′ generate waste heat which, if not dissipated, might heat up the outer shaft 9 at least at or near the distal end 5 of the shaft 3′ inadmissibly, such that tissue coming into contact with the outer shaft 9 would be degraded. For discharging the waste heat generated by the LEDs 8, 8′, heat pipes 10, 10′ are provided thermally coupled at their distal ends to the LEDs 8, 8′, running inside the shaft 3′ until beyond its proximal end 4 and extending into the interior of the head piece 11. The housing 16 of the head piece 11 in conjunction with the shaft 3′ forms a hermetically sealed unit.

Within the head piece 11 the heat pipe 10 is thermally coupled to a Peltier element 15 via a heat transmission block 14 formed of a thermally conductive material, the Peltier element 15 being thermally coupled to the housing 16 of the head piece 11 via a further heat transmission block 14′. The Peltier element 15 is operated such that its side coupled to the heat pipe 10 is cooled and the heat conveyed by the heat pipe 10 plus the waste heat of the Peltier element 15 is transmitted to the housing 16; for this purpose the “cold” side of the Peltier element 15 is thermally coupled to the heat pipe 10, and the “warm” side of the Peltier element 15 is thermally coupled to the housing 16. The further heat pipe 10′ is thermally coupled to the housing 16 only via a heat transmission block 14″. The arrangement shown in FIG. 2 is to be understood only as an example. If only one heat pipe is required for discharging the waste heat generated in the shaft 3′, it may be thermally coupled to the housing 16 via a Peltier element 15, and, possibly, one or more heat transmission blocks 14, 14′, as shown with the heat pipe 10, or may be coupled only via a heat transmission block 14″ to the housing 16, as shown with the heat pipe 10′. If more than one heat pipe is provided for discharging the waste heat generated in the shaft 3′, these may each be coupled to the housing 16 in the same way or in various different ways via one or several Peltier elements or only via one or several heat transmission blocks, according to the requirements pertaining to each case, in particular pertaining to the amount of heat to be transmitted.

In the arrangement shown in FIG. 2 the cooling attachment 20 with the sleeve 21 is shifted over the head piece 11. The sleeve 21 is formed by an outer cylindrical tube 25 the interior side of which carries several cooling bodies 26, 26′ having a soft material into which cooling channels 27, 27′ are integrated through which cooling fluid can flow. The cooling fluid may be a liquid or gaseous medium, in particular water. The cooling bodies 26, 26′ with their interior side 22 abut on the exterior side 12 of the housing 16. The exterior side 12 of the housing 16 and the interior side 22 of the sleeve 21 thus cooperate, forming heat exchange areas for transmission of the heat to be discharged from the head piece 11 to the cooling attachment 20. For this purpose the material of the cooling bodies 26, 26′ has sufficient thermal conductivity.

On that wall of the housing 16 forming a front face of the head piece 11 a plug connector element 13 is arranged, being connected to the housing 16 in a hermetically sealed manner. The housing 16 of the head piece 11 houses an electronic arrangement 17 that is electrically connected to the plug connector element 13 and serves for supply of the Peltier element 15, the LEDs 8, 8′, and the imaging sensor 7. Further, the image data captured by the imaging sensor 7 are transmitted via the electronics arrangement 17. The plug contacts of the plug connector elements 13 are directed in axial direction of the head piece 11, thus by shifting the cooling attachment 20 with its sleeve 21 upon the head piece 11, at the same time the plug connector element 23 of the cooling attachment 20 is connected to the plug connector element 13 of the head piece 11. Electric lines 28, 28′ forming a connection to a supply apparatus (not shown) are connected to the plug connector element 23 of the cooling attachment 20. The electric lines 28, 28′ and the fluid lines 29, 29′ for supply and discharge of cooling fluid run through a supply line 24. The other parts of the cooling fluid supply, such as a pump or a sterile water bag, are not shown in FIG. 2 as well.

Further, in FIG. 2 a locking device 30 is symbolically shown. The locking device may comprise locking elements 32, 32′ arranged on the outer tube 25 and on a holder 31 connected to the endoscope 2′, for example, wherein the locking elements 32, 32′ may be brought into form-fitting engagement with each other and can be detached from each other by a rotation of the endoscope 2′ relative to the cooling attachment 20. In this way, the cooling attachment 20 can be securely connected to and detached from the endoscope 2′.

Instead of the endoscope 2′ the variation of an embodiment of the inventive device 1 as shown in FIG. 2 may comprise a medical instrument (not shown).

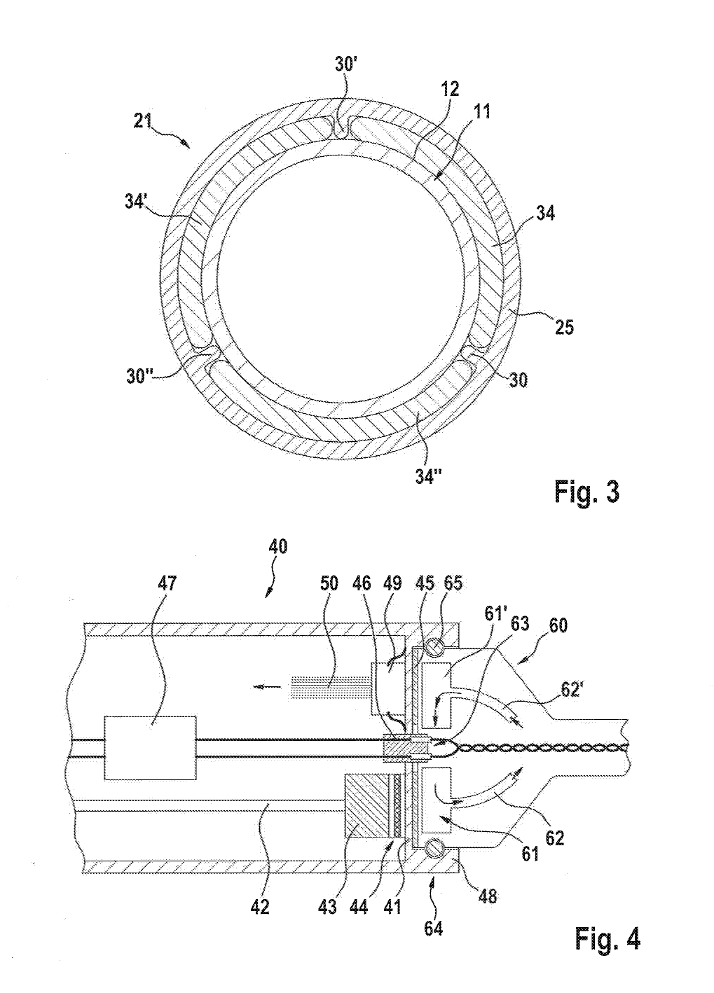

As shown in FIG. 3 in a schematic cross-section, in a second variation of the embodiment shown in FIG. 2, the sleeve 21 may have on its interior side a multiplicity of ridge-shaped insertion noses 33, 33′, 33″ which enable insertion of the head piece 11 into the sleeve 21 such that it fits as accurately as possible, wherein volume variable cooling bodies 34, 34′, 34″ which are formed of an elastic material and which can be filled with cooling fluid are arranged on the interior side of the tube 25 between the insertion noses 33, 33′, 33″. While when not filled with cooling fluid the cooling bodies 34, 34′, 34″ allow easy insertion of the head piece 11 symbolically indicated in FIG. 3, the cooling bodies 34, 34′, 34″ when filled with cooling fluid closely touch the exterior side 12 of the head piece 11. The cooling bodies 34, 34′, 34″ may be configured as balloons as well. The tube 25 may be configured as a gripping shell of a handle or carry such a gripping shell. Relating to other features, the second variation may be designed as the first variation of the first embodiment shown in FIG. 2, but may comprise a medical instrument, for example.

In the first variation of a second embodiment of the invention shown in FIG. 4 in a schematic longitudinal sectional view, the front wall 41 of the head piece 40 acts as a heat exchange area. The waste heat generated by a heat generating component arranged in the distal end section of an endoscope which is configured as described referring to FIG. 2 or in the distal section of a medical instrument, is transmitted via a heat pipe 42 to a Peltier element 44 coupled to the heat pipe 42 via a heat transmission block 43, the Peltier element 44 being thermally coupled to the front wall 41; since the front wall 41 is designed flat, the Peltier element 44 can be directly coupled to the front wall 41. At least on a part of the front wall 41 a layer 45 of a thermally conductive elastic material is applied. The cooling attachment 60 has a heat exchange chamber 61 which through which cooling fluid can flow and which is arranged opposite to that zone of the front wall 41 to which the Peltier element 44 is coupled. The heat exchange chamber 61 can be connected to one or more further heat exchange chambers 61′ arranged opposite to other zones of the front wall 41 via a line (not shown for clarity) and can be supplied via fluid lines 62, 62′ by a supply apparatus (not shown), a possible direction of flow being indicated by the arrows. Heat is exchanged from the front wall 41 into the cooling fluid via the layer 45 to the heat exchange chambers 60, 61′ from which the heat is discharged by the cooling fluid. Further, the front wall 41 carries a plug connector element 46 cooperating with a plug connector element 63 of the cooling attachment 60 for establishing an electric connection between an electronics arrangement 46 and the supply apparatus, as described above referring to FIGS. 1 and 2. A locking device 64 is illustrated symbolically in FIG. 4, comprising locking elements 65 which releasably engage with recesses on the interior side of an axial extension 48 of the cylindrical housing of the head piece 40 and on the exterior side of the cooling attachment 60. The locking device 64 permits fixing the cooling attachment 60 to the head piece 40 and exerting a pressing force onto the front wall 41 sufficient for heat exchange. Locking tolerances are compensated by the layer 45.

Instead of or in addition to a heat source arranged in the distal end section of the endoscope and/or of the medical instrument a heat source, for example an LED 49, may be arranged in the head piece 40. In an endoscope with an LED 49 arranged in the head piece 40, a glass fiber optics 50 is provided for transmitting the light generated by the LED 49 to the distal end of the shaft and for illuminating the endoscopic scene (indicated by the arrow). The LED 49 may be arranged directly at and thermally coupled to the front wall 41. In a medical instrument instead of the LED 49 a motor or another heat generating component may be present, for example, which also is thermally coupled to the front wall 41. Instead of the light guide 50 in this case a drive shaft may be provided for transmitting the motion generated by the motor into the distal section of the instrument, for example. The waste heat generated by the heat generating component, in particular by the LED 49, is discharged by the further heat exchange chamber 61′. It is to be understood that the depiction in FIG. 4 only in an exemplary manner shows an arrangement with a heat pipe 42 for transmission of the waste heat generated in the distal end section of the endoscope to the head piece 40 and with a light and/or heat source (LED 49) arranged in the head piece 40. Thus, it is as well conceivable, for example, that an endoscope or a medical instrument only has light and/or heat sources in its distal end section or only light and/or heat sources in the head piece 40, including corresponding heat discharge in each case. The same also pertains to the first embodiment of the invention shown in FIGS. 1 through 3.

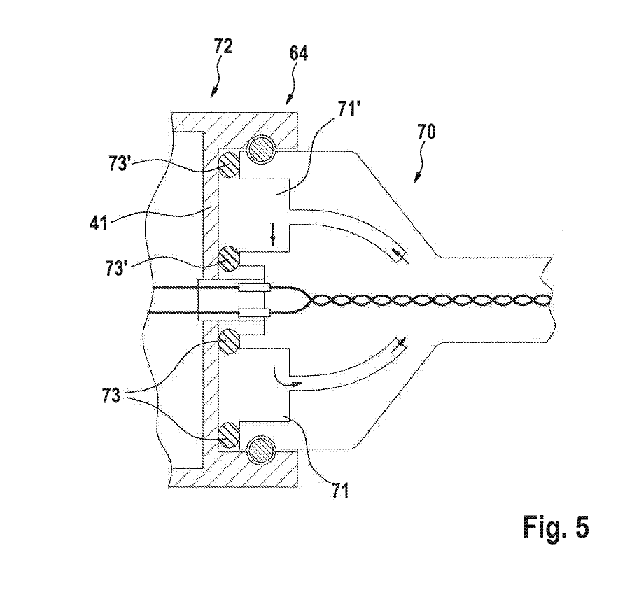

FIG. 5 shows a second variation of the second embodiment of the invention. In this embodiment, instead of the heat exchange chambers 61, 61′ provided in the first variation in accordance with FIG. 4, fluid chambers 71, 71′ are provided that are open towards the front wall 41. By attaching the cooling attachment 70 to the head piece 72 the fluid chambers are enclosed in a fluid-tight manner by the front wall 41 itself and by seals enclosing the fluid chambers 71, 71′ laterally in a fluid-tight manner. The seals may be seal rings 73, 73′, for example. In this case the cooling fluid gets in direct contact with the metallic front wall 41; a thermally conductive layer of elastic material on the front wall 41 can be dispensed with in this case. Relating to other features, the cooling attachment 70 and the head piece 72 are designed like the cooling attachment 60 and the head piece 40 as described referring to FIG. 4, the locking device 64 permitting exerting a pressing force to the front wall 41 sufficient for the sealing effect of the seals.

For clarity, not all reference numerals are shown in all figures. Reference numerals not explained with respect to one figure have the same meaning as in the other figures.

The invention being thus described, it will be obvious that the same may be varied in many ways. Such variations are not to be regarded as a departure from the spirit and scope of the invention, and all such modifications as would be obvious to one skilled in the art are to be included within the scope of the following claims.

1. A medical device comprising:
a medical instrument having a head piece;
at least one heat generating component arranged in the medical instrument;
a heat exchange area provided in the head piece for discharging heat generated by the at least one heat generating component to an exterior of the medical instrument; and
a cooling attachment connectable to the medical instrument, the cooling attachment being configured for absorbing heat from the heat exchange area of the head piece and for dissipating heat via a cooling fluid flowing through the cooling attachment.

2. The medical device according to claim 1, wherein the medical device is an endoscopic device.

3. The medical device according to claim 1, wherein the medical instrument is an endoscope.

4. The device according to claim 2, wherein the endoscope device has an elongate shaft, wherein the head piece is arranged at a proximal end section of the elongate shaft, and wherein in a distal end section of the elongate shaft the at least one heat generating component is arranged and within the elongate shaft a heat transmitter for transmitting the heat generated by the at least one heat generating component to the heat exchange area of the head piece is arranged.

5. The device according to claim 1, wherein the medical instrument is hermetically sealed.

6. The device according to claim 1, wherein the cooling attachment is a handle.

7. The device according to claim 1, wherein the cooling attachment is designed for single use.

8. The device according to claim 1, wherein the cooling attachment is configured such that the cooling fluid is routed in a closed cooling circuit therethrough.

9. The device according to claim 1, wherein the cooling attachment is configured such that the cooling fluid flows therethrough under the effect of gravity.

10. The device according to claim 1, wherein the cooling attachment has a heat exchange area that is adapted to be at least partially brought into thermal contact to the heat exchange area of the head piece and which is thermally coupled to a heat exchanger through which the cooling fluid is configured to flow.

11. The device according to claim 1, wherein the heat exchange area of the head piece is a radially exterior surface.

12. The device according to claim 1, wherein the heat exchange area of the head piece forms at least a part of a proximal front face of the head piece.

13. The device according to claim 1, wherein a thermally conductive material is arranged in a gap remaining after connecting the cooling attachment to the medical instrument between the heat exchange area of the head piece and a heat exchange area of the cooling attachment, the thermally conductive material being subject to a volume increase sufficient for filling the gap in heating up during operation of the device.

14. The device according to claim 1, wherein, in a gap remaining after connecting the cooling attachment to the medical instrument between the heat exchange area of the head piece and a surface of the cooling attachment, at least one volume variable element is arranged that is fillable with cooling fluid.

15. The device according to claim 1, wherein the cooling attachment is configured for forming a fluid-tight connection to the head piece such that between the heat exchange area of the head piece and a surface of the cooling attachment, a space is formed that is adapted to have the cooling fluid flow therethrough.

16. The device according to claim 1, wherein the head piece and the cooling attachment have plug connector elements cooperating to establish an electric connection between the medical instrument and the cooling attachment.

17. A cooling device for an endoscope and/or a medical instrument, wherein the endoscope and/or the medical instrument, respectively, has a head piece, wherein in the endoscope and/or in the medical instrument, respectively, at least one heat generating component is arranged, and wherein the head piece has a heat exchange area for discharging the heat generated by the at least one heat generating component to an exterior of the endoscope and/or the medical instrument, respectively, wherein the cooling device is configured as a cooling attachment connectable to the endoscope and/or the medical instrument, respectively, the cooling attachment being configured for absorbing heat from the heat exchange area of the head piece and for dissipating heat via a cooling fluid flowing through the cooling attachment.

18. A device comprising:
an endoscope having a head piece;
at least one heat generating component arranged in the endoscope;
a heat exchange area provided in the head piece for discharging heat generated by the at least one heat generating component to an exterior of the endoscope; and
a cooling attachment connectable to endoscope, the cooling attachment being configured for absorbing heat from the heat exchange area of the head piece and for dissipating heat via a cooling fluid flowing through the cooling attachment.

19. The device according to claim 18, wherein the endoscope is a medical endoscope.
